(19) United States
(12) Patent Application Publication
(10) Pub. No.: US 2022/0068449 A1
(43) Pub. Date: Mar. 3, 2022

(54) INTEGRATED DIAGNOSTICS SYSTEMS AND METHODS

(71) Applicant: KONINKLIJKE PHILIPS N.V., EINDHOVEN (NL)

(72) Inventors: PRESCOTT KLASSEN, CAMBRIDGE, MA (US); SANDEEP MADHUKAR DALAL, WINCHESTER, MA (US); SAMUEL FRANK PILATO, CAMBRIDGE, MA (US); VADIRAJ HOMBAL, WAKEFIELD, MA (US)

(21) Appl. No.: 17/415,048

(22) PCT Filed: Dec. 13, 2019
(86) PCT No.: PCT/EP2019/085006

§ 371 Date: Jun. 17, 2021

Related U.S. Application Data
(60) Provisional application No. 62/782,401, filed on Dec. 20, 2018.

(51) Int. Cl. G16H 15/00; G06F 40/30; G06F 40/40; G16H 30/20; G16H 10/60

(52) U.S. Cl. CPC G16H 15/00; G06F 40/30; G16H 10/60; G16H 30/20; G06F 40/40

(57) ABSTRACT

A medical information technology (IT) system includes a plurality of medical information systems storing medical reports in different respective medical information system-specific medical report formats. An integrated diagnostics system includes: a medical report transform operative to transform text of medical reports stored in the different respective system report formats to an integrated diagnostics representation which represents the text of the medical reports as vocabulary category values of a vocabulary of categories; and a plurality of document processing tasks each operative to invoke the medical report transform to transform one or more medical reports processed by the task to the integrated diagnostics representation and to perform the task on the vocabulary category values of the integrated diagnostics representation of the one or more medical reports processed by the task.

### FIELD

The following relates generally to the medical diagnostics arts, medical imaging arts, pathology arts, medical facility information technology (IT) infrastructure arts, and related arts.

### BACKGROUND

An IT infrastructure for a hospital or other medical facility typically includes a number of different systems. For example, to support radiology/medical imaging practices, the IT infrastructure may include: a Picture Archiving and Communication System (PACS) providing infrastructure for storage and distribution of medical images; a Radiology Information System (RIS) providing infrastructure for patient scheduling of imaging examinations, storage and distribution of radiology reports, and so forth; a Pathology Information System (PIS) providing similar services for pathology laboratories and services; IT systems for medical specializations such as a Cardiovascular Information System (CVIS) providing infrastructure for storage and distribution of cardiology data; an Electronic Health Record (EHR), Electronic Medical Record (EMR), or otherwise-named infrastructure providing for storage and distribution of patient medical history information; and so forth.

These IT divisions have strong practical benefits. However, in actual practice, the treatment of a patient usually intersects numerous different IT systems. For example, records for a single cancer patient may include: medical images stored on the PACS; radiology reports stored on the RIS; pathology reports stored on the PIS; routine electrocardiograph (ECG) tests or other cardiac test data stored on the CVIS; as well as summaries of physical examinations performed by the patient's general practice (GP) physician and oncologist along with high level summaries of the imaging, pathology, ECG, or other specialized medical tests all stored on the EHR or EMR. Clinicians may become adept at using these various IT systems; yet, the potential for missing important links between findings, recommendations, clinician impressions, and the like stored on these diverse medical IT systems is an ever-present concern. Indeed, it is even possible for a clinician to miss such links within a single area of specialization. For example, a radiologist who reads many radiology examinations over an eight-hour (or longer) work shift may record a finding in the description section of a radiology report on a radiology examination, but forget to include a corresponding recommendation to perform a pathology examination suggested by the finding in the recommendations section of the report.

A further difficulty with the diversity of different medical IT systems is that it makes it difficult to provide a combined summary of patient data stored on these various systems. It is especially difficult provide such a summary in layperson's terms, for example in the form of a report that is readily comprehensible by the (lay) patient.

The following discloses certain improvements.

### SUMMARY

In some non-limiting illustrative embodiments disclosed herein, a medical information technology (IT) system comprises one or more computers and one or more data storage media. The one or more computers and the one or more data storage media are interconnected by an electronic network, and the one or more data storage media store instructions executable by the one or more computers to define a plurality of medical information systems storing medical reports in different respective medical information system-specific medical report formats, and an integrated diagnostics system. For example, the plurality of medical information systems may include a Pathology Information System (PIS) storing pathology reports in a pathology report format and/or a Radiology Information System (RIS) storing medical imaging examination reports in a medical imaging examination report format that is different from the pathology report format. The integrated diagnostics system includes: a medical report transform operative to transform text of medical reports stored in the different respective system report formats to an integrated diagnostics representation which represents the text of the medical reports as vocabulary category values of a vocabulary of categories; and a plurality of document processing tasks each operative to invoke the medical report transform to transform one or more medical reports processed by the task to the integrated diagnostics representation and to perform the task on the vocabulary category values of the integrated diagnostics representation of the one or more medical reports processed by the task.

In some non-limiting illustrative embodiments disclosed herein, a non-transitory storage medium stores instructions which are readable and executable by one or more computers to extract textual content from a medical imaging examination report on a medical imaging examination of a patient, and add metadata describing the textual content extracted from the medical imaging examination report to an image of the medical imaging examination of the patient. The added metadata describing the textual content extracted from the medical imaging examination report includes a hyperlink to the medical imaging examination report.

In some non-limiting illustrative embodiments disclosed herein, a method is performed in conjunction with a Pathology Information System (PIS) which stores pathology reports in a pathology report format, and a Radiology Information System (RIS) which stores medical imaging examination reports in a medical imaging examination report format that is different from the pathology report format. The method comprises, using an electronic processor programmed by instructions stored on a non-transitory storage medium: converting at least one pathology report and at least one medical imaging examination report to an integrated diagnostics representation which represents the text of the converted reports as vocabulary category values of a vocabulary of categories; temporally ordering the converted reports based on timestamps of the respective reports; identifying a responsive report and a causational report based on vocabulary category values of the converted responsive report being responsive to vocabulary category values of the converted causational report; and displaying, on a workstation, a summary of the vocabulary category values used in the identifying.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a hospital information technology (IT) system as described herein.

FIG. 2 diagrammatically shows an illustrative embodiment of the medical report transform (IDRx) of the IT system of FIG. 1.

FIG. 3 diagrammatically shows an illustrative embodiment of the medical image tagging task of the IT system of FIG. 1.

FIG. 4 diagrammatically shows an illustrative embodiment of the report tracking task of the IT system of FIG. 1.

FIG. 5 diagrammatically shows an example a radiology pathology concordance dashboard produced by the report tracking task of FIGS. 1 and 4.

FIG. 6 diagrammatically shows an illustrative embodiment of the recommendation inference task of the IT system of FIG. 1.

FIG. 7 diagrammatically shows an illustrative embodiment of the impression inference task of the IT system of FIG. 1.

FIG. 8 diagrammatically shows an illustrative implementation of the patient timeline task of the IT system of FIG. 1.

FIG. 9 diagrammatically shows a vocabulary outline, in which elements and attributes that most heavily serve discourse/pragmatic roles in developing the patient timeline are underlined.

FIG. 10 diagrammatically shows an illustrative embodiment of the outliers detection task of the IT system of FIG. 1.

### DETAILED DESCRIPTION

One possible approach for addressing difficulties caused by the use of a number of different medical IT systems at a hospital or other medical facility might be to replace them by a single comprehensive medical IT system that provides services for medical imaging/radiology, pathology, cardiology and other medical specialties, as well as providing an electronic patient medical history. However, this may not be an ideal solution. Such an extensive upgrade may not be practical for an existing hospital or other medical institution as it would likely cause a massive disruption in ongoing patient record-keeping. Furthermore, the resulting comprehensive medical IT system might be cumbersome to use. For example, the user interface (UI) of an RIS is tailored to the needs of radiology and medical imaging laboratories; the UI of a PIS is tailored to the needs of pathology laboratories; and so forth. A comprehensive medical IT system would have the potential of introducing extraneous user input fields, options, and features for users operating in different medical areas, which would lead to inefficiencies and possibly data entry and/or reading errors. Likewise, the report formats, metadata formatting, and other data structures used to store medical imaging data and reports is very different from the data structures used to store pathology data, which is different again from the data structures used to store cardiology data, and so forth. Still further, a comprehensive medical IT system might introduce data management and security issues, as for example a radiologist may be given access to medical data in areas in which the radiologist is not fully qualified. Still yet further, different medical IT system vendors may be well-regarded in different areas, so that the hospital or other medical institution is motivated to employ a PIS from one vendor who specializes in pathology information systems, and an RIS from another vendor who specializes in radiology information system, and so forth.

In systems and methods disclosed herein, the existing medical IT infrastructure paradigm of employing separate systems for different medical areas (e.g. PACS, RIS, PIS, CVIS, EHR . . . ) is retained; but these systems are augmented by an integrated diagnostics system that provides automated and controlled cross-fertilization of data between the different systems. This is achieved by the use of an integrated diagnostics representation (IDR) which represents medical reports and other text-based medical data using a standardized vocabulary of categories. Thus, for example, a radiologist continues to produce radiology reports in the RIS environment using a radiology-specific reporting format, the pathologist continues to product pathology reports in the PIS environment using a pathology-specific (or even finer-grained pathology lab-specific) reporting format, and so forth; but, an integrated diagnostics representation extractor (IDRx) converts each of these documents to a representation in which key concepts (represented by categories of the vocabulary of categories) are extracted out to form IDR representations of the documents. In this way, for example, a recommendation to perform a pathology test contained in a radiology report is easily linked with a corresponding pathology report that summarizes the results of the recommended pathology test.

As further disclosed herein, a wide range of integrated diagnostics tasks can be implemented by leveraging the disclosed IDR representation. For example, IDR category:value pairs extracted from a radiology report in the RIS summarizing an imaging examination can be used as metadata tags for the medical image(s) stored in the PACS which were read to produce that radiology report, so that this information is available to any clinician who retrieves the image(s) from the PACS for viewing. Moreover, the use of a common vocabulary of categories enables a subsequent report for the same patient from a different medical field to also be annotated to the image(s), for example by matching an IDR recommendation:value from the radiology report on the image(s) with an IDR examtype:value from a pathology report. Another illustrative integrated diagnostics task enabled by the disclosed IDR representation is a cross-area correlation task generating concordance/discordance information between related document in different medical areas (e.g. determining concordance and/or discordance of findings of a radiology examination with findings of a related pathology report for the same patient). Other illustrative enabled tasks provide authoring assistance to clinicians (e.g., when the radiologist dictates a finding into a radiology report under draft, the extracted IDR finding:value can be used to identify one or more likely recommendations and/or impressions that commonly co-occur with that finding:value pair and the identified recommendations and/or impressions can be suggested in the radiology UI for inclusion in the respective recommendations and/or impressions section of the radiology report). Another illustrative enabled task detects outliers in reports under draft, e.g. entry of a critical finding without a corresponding alert that is usually included when that critical finding is dictated. Still yet another illustrative enabled task is a timeline tracking task which summarizes and combines information from different medical areas into a timeline for a specific medical patient, so as to provide a coherent discourse (preferably in terminology comprehensible by a lay person) summarizing and contextualizing the patient's medical journey to date.

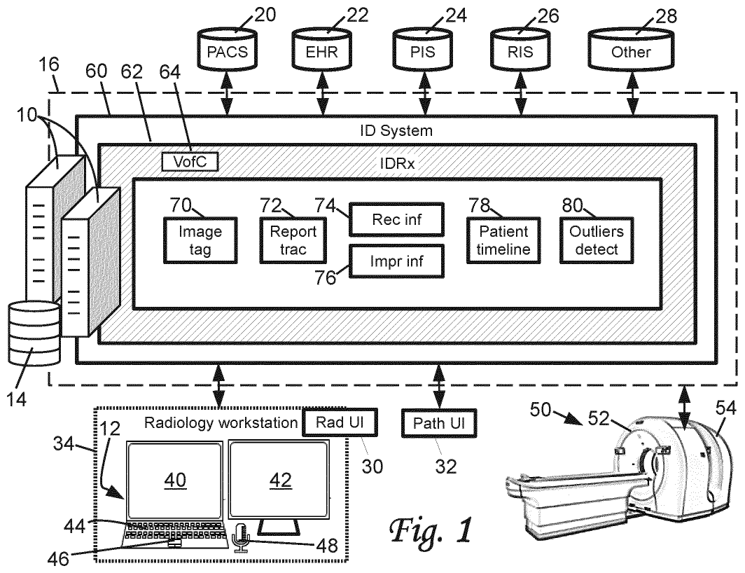

With reference to FIG. 1, a hospital information technology (IT) system includes one or more computers 10, 12 and one or more data storage media 14. The computers 10, 12 and data storage media are interconnected by an electronic network 16 (diagrammatically indicated in FIG. 1) to define a Picture Archiving and Communication System (PACS) 20 storing medical images, an electronic health record (EHR) 22 storing patient histories and related data (the EHR 22 is commonly referred to in the art by various similar nomenclatures such as an Electronic Medical Record, EMR, or so forth; the illustrative EHR 22 is intended to encompass such variants), and a plurality of medical information systems 24, 26, 28 storing medical reports in different respective medical information system-specific medical report formats. The diagrammatically illustrated medical information systems include a Pathology Information System (PIS) 24 storing pathology reports in a pathology report format, and a Radiology Information System (RIS) 26 storing medical imaging examination reports in a medical imaging examination report format that is different from the pathology report format. (It is noted that specific medical information systems may employ alternative report nomenclature; for example, some RIS 26 refer to the medical imaging examination reports as radiology reports, while pathology reports may be named based on the type of pathology examination, e.g. a biopsy report. The terms employed herein, e.g. medical imaging examination report and pathology report, are to be understood as encompassing these alternative nomenclatures). The diagrammatically indicated other medical information systems 28 may, by way of further example, include a Cardiovascular Information System (CVIS) or other specialty-specific medical information system.

The PACS 20, EHR 22, and various medical information systems 24, 26, 28 are diagrammatically shown in FIG. 1, but are to be understood as being implemented by suitable instructions stored on the non-transitory storage media 14 and readable and executable by the computer(s) 10, 12 to cause the computers to define or implement each such system 20, 22, 24, 26, 28. This implementation includes providing suitable user interface(s) (UI) 30, 32 for user access by medical personnel, clerical personnel, or other authorized (e.g. logged-in) users to input data to the systems, retrieve data from the systems, modify data on the systems, or so forth. The UI are typically implemented on various workstations 34. By way of non-limiting illustration, FIG. 1 shows an illustrative radiology UI 30 presented on an illustrative radiology workstation 34 for user interfacing with the RIS 26 and PACS 20, and a diagrammatically indicated pathology UI 32 implemented on a pathology laboratory workstation (not shown). Various other UI may be implemented on various other types of workstations (not shown in FIG. 1), such as a physician UI implemented on a physician's office or personal computer (at the hospital and/or remotely via VPN or other remote UI access protocols) or so forth. Each workstation (such as the illustrative radiology workstation 34) typically includes one or more displays 40, 42 for displaying appropriate medical information (e.g. text of medical reports, medical images retrieved from the PACS 20, or so forth as appropriate for the particular system UI), and one or more user input devices 44, 46, 48 such as an illustrative keyboard 44, trackpad 46 (or mouse, trackball, or other pointing device), dictation microphone 48, and/or so forth. The particular display and user input components of a given workstation depend upon various factors such as cost considerations, needs of the user, types of data presented and manipulated by the UI, and/or so forth. For example, the illustrative radiology workstation 34 includes two displays 40, 42 as this is often useful for a radiologist who may use one display for showing medical images and the other display for presenting text of a radiology report under draft, and a dictation microphone as such is commonly used by radiologists for dictating a radiology report. On the other hand, a pathology laboratory workstation may employ less sophisticated display technology if pathology lab workers are expected to be inputting text but not viewing high resolution medical images. A general practitioner (GP) physician's workstation may have broader access to the various systems 20, 22, 24, 26, 28 as may be required for the diverse types of medical data the GP physician is expected to review; whereas, a radiology technician's workstation UI may be authorized (e.g. logged into) only the PACS 20 and perhaps the RIS 26 as these may be the only systems a radiology technician needs to access.

The medical IT system typically is connected by the network 16 with various medical devices, such as an illustrative medical imaging device 50 and a wide range of other medical devices (not shown) such as patient monitors, mechanical ventilation systems, and/or so forth. The illustrative medical imaging device 50 is a positron emission tomography/computed tomography (PET/CT) scanner including a CT gantry 52 and a PET gantry 54, a configuration commonly used for tasks such as oncology imaging, brain imaging, cardiac imaging, and/or so forth. Other non-limiting examples of medical imaging devices include magnetic resonance imaging (MRI) scanners, fluoroscopy imaging devices, ultrasound (US) imaging devices, standalone CT scanners, and/or so forth. The various medical imaging devices may be variously organized, e.g. at radiology laboratories which may be on-site (that is, hospital facilities) and/or off-site (e.g. third-party imaging laboratories or services), and are connected via the network 16 to operatively interact with the PACS 20 to store the generated medical images.

The various systems 20, 22, 24, 26, 28 are illustrated as separate systems. However, certain of these may, in a medical IT system of a specific hospital or medical institution, be combined. For example, the PACS and RIS may be combined as an integrated PACS/RIS system storing both medical images and medical imaging examination reports. As previously discussed, however, there are substantial advantages to segregating the various medical IT tasks into various systems, and especially for segregating medical information systems for largely unrelated medical specialties such as radiology (employing the RIS 26) and pathology (employing the PIS 24).

The one or more computers 10, 12 typically include one or more server computers 10. If there are two or more server computers 10, they may be variously configured to share the computing workload, such as different server computers being dedicated to perform distinct processing (for example, one server computer may handle the PACS 20 and another the EHR 22); or, the server computers may be interconnected by the network 16 to form a computing cluster, cloud computing resource, or so forth where the various server computers 10 operate cooperatively to implement a single system such as the PACS 20; or so forth. Certain operations of the various systems 20, 22, 24, 26, 28 may be performed at the computer 12 of a workstation 34, such as local implementation of generating the UI display at the workstation 34, for example. Again, varying degrees of sharing or distribution of processing between the server computer(s) 10 and the local workstations 12 is contemplated. It will be further appreciated that the network 16 can be implemented using various networking technologies (e.g. wired, wireless, or combined; various transmission modulation technologies, and so forth), various networking protocols (e.g. WiFi, Ethernet, or so forth), various network components (a local area network, a WiFi network, the Internet, and/or so forth), and that a given communication link over the network 16 may involve various of these technologies, protocols, and components.

With continuing reference to FIG. 1, the various medical information systems 24, 26, 28 store medical reports in different respective medical information system-specific medical report formats, e.g. the PIS 24 stores pathology reports in a pathology report format, the RIS 26 stores medical imaging examination reports in a medical imaging examination report format that is different from the pathology report format, and so forth. These various system-specific medical report formats are preferably optimized for efficient data entry, storage, and display of the type(s) of medical report(s) handled by that system. For example, the pathology report format employed by the PIS 24 preferably includes fields or other data structures for storing pathology report-specific information such as biopsy sample identification information, sample stain information (if any), pathologist observations as to pathology features, and so forth; while, the RIS 26 preferably includes fields or other data structures for storing medical imaging examination report-specific information such as imaging modality, imaging field of view, contrast agent employed (if any), tumor dimensions, and so forth. Because the different medical information systems 24, 26, 28 generally store medical reports in different system-specific medical report formats, and in some specific implementations may be provided by different medical IT system vendors using different vendor-specific formats, combining diagnostic information from the different systems is difficult. Conventionally integration of diagnostics from different systems has been primarily a manual process, e.g. a GP physician working at an office workstation may open a window on the workstation to access the RIS and bring up a medical imaging examination report on a patient, and open another window to access the PIS to bring up a pathology report on the patient. On the other hand, a pathology lab technician may not be authorized to access the RIS at all, and hence may be unable to read a medical imaging examination report on a patient, or view the underlying images from the PACS, to provide imaging information that might be useful in performing the pathology examination. Even if such access is authorized, the pathology lab technician may be unfamiliar with the RIS or PACS and hence be unable to retrieve the imaging report or underlying images.

To facilitate diagnostics that integrate the medical reports stored in the various medical information systems 24, 26, 28, the medical IT system of FIG. 1 includes an integrated diagnostics (ID) system 60 which includes a medical report transform (sometimes denoted herein by the shorthand “IDRx”) 62 that is operative to transform text of medical reports stored in the different respective system report formats to an integrated diagnostics representation which represents the text of the medical reports as vocabulary category values of a vocabulary of categories 64. The ID system 60 further includes a plurality of document processing tasks 70, 72, 74, 76, 78, 80. Each document processing task is operative to invoke the medical report transform 62 to transform one or more medical reports processed by the task to the integrated diagnostics representation and to perform the task on the vocabulary category values of the integrated diagnostics representation of the one or more medical reports processed by the task. The illustrative document processing tasks include: a medical image tagging task 70; a report tracking task 72; a recommendation inference task 74; an impressions inference task 76; a patient timeline task 78; and an outliers detection task 80. These are merely illustrative document processing tasks which may be implemented by the ID system 60, and fewer, additional, and/or different document processing tasks may be implemented that advantageously leverage the medical report transform 62 to convert one or more medial reports to the common integrated diagnostics representation.

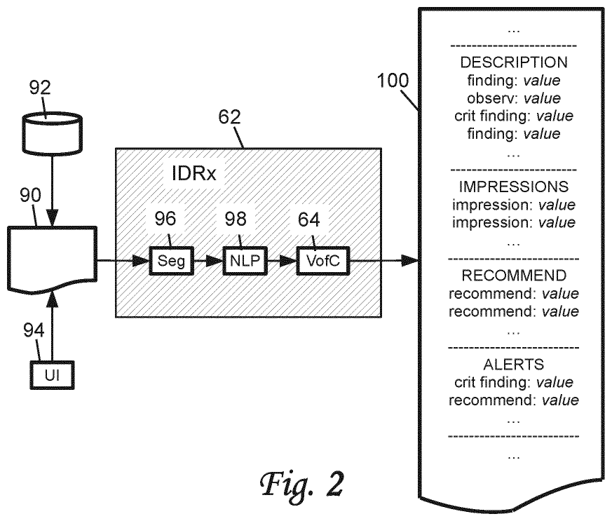

With reference to FIG. 2, an illustrative embodiment of the medical report transform (IDRx) 62 is shown. A medical report 90 is received by the medical report transform 62. The medical report 90 may be retrieved from a medical information system 92, e.g. from one of the medical information systems 24, 26, 28, having been invoked by one of the tasks 70, 72, 74, 76, 78, 80 to transform the medical report 90. Alternatively, the medical report 90 may be medical report under draft by a user operating a UI 94 of one of the medical information systems 24, 26, 28, for example a medical imaging examination report under draft by a radiologist operating the radiology UI 30 via the radiology workstation 34, or a pathology report under draft by a pathology lab technician operating the pathology UI 32 via a pathology workstation (not shown in FIG. 1), or so forth. The illustrative medical report transform 62 processes the medical report 90 by report segmentation 96 that segments the report into identifiable sections based on header text and/or based on an a priori known report schema identifying the report sections for the report format employed by the sourcing medical information system. For example, a medical imaging examination report typically has standard sections defined by the RIS 26. One non-limiting section schema for a medical imaging examination report may include a “Description” section setting forth the radiologist's findings (e.g., identifying a tumor); an “Impressions” section setting forth the radiologist's impressions (e.g., qualitative and/or quantitative characteristics of the tumor such as size and/or geometry); a “Diagnosis” section setting forth any diagnoses determined by the radiologist (e.g., a Breast Imaging-Reporting and Data System (BI-RADS) score for a tumor identified in a mammogram); and a “Recommendations” section setting forth any follow-up recommendations the radiologist may provide (e.g., a recommendation to perform a biopsy on a suspicious tumor identified in the report). This is merely an illustrative section schema, and additional or fewer and/or other sections may be employed; additionally or alternatively, the report segmentation process may use the textual content of report headers and/or report content to perform the segmentation into sections.

The textual content of the sections of the report are then analyzed by natural language processing (NLP) 98 which may, for example, employ a sequence of a tokenizer that identifies white space to break text into individual tokens (typically words or numbers), a grammatical parser that parses the tokens into grammatical units (e.g. sentences) and parts-of-speech (e.g. nouns, verbs, adjectives, and so forth), a phrase structure parser, dependency parser, named entity recognizer, semantic role labeler, and/or other NLP token grouping/classification. In one approach, an ensemble of rule-based and ML components are trained based on labeled and unlabeled clinical text collected from past (i.e. historical) radiology reports and curated to create an overall model of clinical report text. Elements of the natural language processing may include words, sentences, part-of-speech labels, and other basic linguistic information, as well as higher level semantic structures that identify units of text as values of vocabulary categories such as findings, diagnosis, follow-up recommendations, and other elements of discourse found in clinical text. The parsed text is then searched to identify vocabulary category values of a vocabulary of categories 64. The vocabulary of categories is a closed set of categories. These are categories of medical language commonly used in medical reports, such as (by way of non-limiting illustrative example): “finding”, “critical finding”, “recommendation”, “biopsy sample”, “reason for exam”, “diagnosis”, “impression”, and “observation”, and/or other vocabulary categories. A vocabulary category is instantiated in a particular medical report by a value for that category, e.g. a possible value for the “diagnosis” vocabulary category could be “prostate cancer”. The vocabulary could be hierarchical whereby all values belonging to a sub-category necessarily also belong to a higher-up category, e.g. all values that belong to the (sub-)category “critical diagnosis” also belong to the higher-up category “diagnosis”. Likewise, a value belonging to the (sub-)category “follow-up recommendation” necessarily also belongs to the higher-up category “recommendation”. There may also be some overlap between categories of the vocabulary, e.g. terms that fall within the “impression” category may also fall into the “finding” category. Alternatively, the vocabulary of categories may be designed to be mutually exclusive so that there is no such overlap.

By way of non-limiting illustration, FIG. 2 diagrammatically shows a possible example of the medical report 90 transformed into the integrated diagnostics representation 100, with report sections labeled by the headings “DESCRIPTION”, “IMPRESSIONS”, “RECOMMEND(ations)”, and “ALERTS”, and the content of each section represented as (vocabulary category:value) pairs, e.g. “finding:value”, “observ(ation):value”, et cetera.

In the following, the various non-limiting illustrative tasks 70, 72, 74, 76, 78, 80 are described.

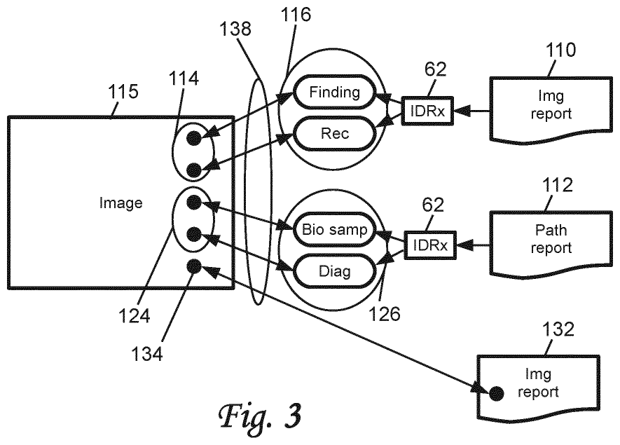

With reference to FIG. 3, the medical image tagging task 70 is defined by instructions stored on the one or more data storage media 14 and executable by the one or more computers 10, 12 to identify a medical imaging examination report 110 on a medical imaging examination of a patient. The medical report transform 62 is invoked to transform the identified medical imaging examination report 110 to the integrated diagnostics representation. Metadata 114 is added to an image 112 of the medical imaging examination of the patient stored on the PACS 20. The metadata 114 describes one or more vocabulary category values 116 of the identified and transformed medical imaging examination report.

Optionally, the medical image tagging task 70 may further identify a pathology report 120 on the (same) patient, and invoke the medical report transform 62 to transform the identified pathology report 120 to the integrated diagnostics representation. One or more correlated vocabulary category values 126 are located in the identified and transformed pathology report that correlate with the one or more vocabulary category values 116 described by the metadata 114 added to the image 110, and further metadata 124 is added to the image 115 describing the one or more correlated vocabulary category values in the identified and transformed pathology report. The metadata vocabulary category values 126 from the pathology report 112 are correlated with the vocabulary category values 116 from the medical imaging examination report 110 based on a structure or schema of the vocabulary of categories 64. For example, a finding:tumor (marker) vocabulary category value identified in the imaging examination report 110 (where (marker) is a marker in the image 115 applied by the radiologist during the drafting of the medical imaging examination report 110 to mark the tumor in the image) is correlated with a Diagnosis:malignant vocabulary category value located in the pathology report 112.

Optionally, the tagging can continue through further follow-up medical reports, such as a follow-up medical imaging examination report 132 that generates further vocabulary category value that correlate with the existing metadata 114, 124 and are suitably added as further metadata 134. For example, the follow-up medical imaging examination report 132 may be performed after administration of one or more rounds of chemotherapy in order to assess efficacy of that therapy, and the further metadata 134 may be descriptive of an impression or observation as to whether the tumor size has increased, decreased, or remained unchanged.

In some embodiments, the added metadata 114, 124, 134 are Digital Imaging and Communications in Medicine (DICOM) metadata, which is a common metadata format for metadata annotated to images in a PACS. In some embodiments, the added metadata 114, 124, 134 includes hyperlinks 138 to the respective medical reports 110, 112, 132 that are transformed to obtain the one or more vocabulary category values 116, 126 described by the metadata 114, 124. The hyperlinks 138 may be to the report generally, or may be to the specific section of the report containing the vocabulary category value, or even to the specific sentence or other finer-grained unit of the report containing the vocabulary category value. By “hyperlink” it is meant that when the metadata is displayed, there is a selectable element of the metadata that, if selected (e.g. by a mouse click) causes the UI displaying the image 115 to bring up the report (or the section of the report containing the vocabulary category value). By way of the added metadata 114, 124, 134, anyone with access to the PACS 20 can review the information from the respective medical reports 110, 112, 132 that is described by the metadata 114, 124, 134. If the hyperlinks 138 are included as part of this metadata, then the user can also easily bring up the source medical report. (If the particular user has access to the PACS 20 but not to the medical information system containing the linked report, then preferably the user receives a message indicating that access to the report is denied).

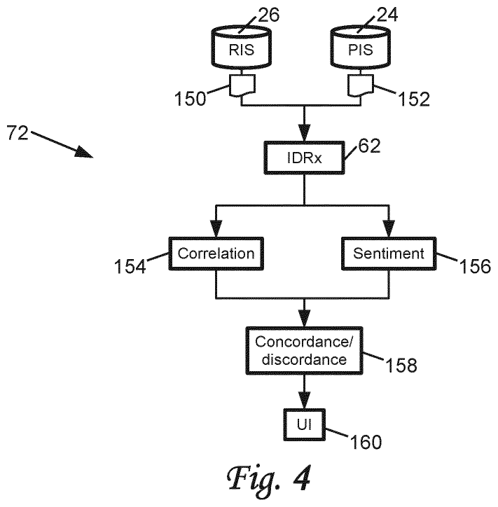

With reference to FIG. 4, the report tracking task 72 is defined by instructions stored on the one or more data storage media 14 and executable by the one or more computers 10, 12 to identify a first medical report 150 on a patient in a first medical information system (e.g., the illustrative RIS 26) which is stored in a first system report format (e.g., the RIS report format). The medical report transform (IDRx) 62 is invoked to transform the first medical report 150 to the integrated diagnostics representation, and one or more first report vocabulary category values are identified in the transformed first medical report. Similarly, a second medical report 152 on the patient is identified in a second medical information system (e.g., the illustrative PIS 24) which is stored in a second system report format (e.g. the PIS report format) that is different from the first (e.g. RIS) system report format. The medical report transform (IDRx) 62 is invoked to transform the second medical report 152 to the integrated diagnostics representation, and or more second vocabulary category values in the transformed second medical report are identified that correlate with the first report vocabulary category values. The report correlation process 154 is suitably based on a structure or schema of the vocabulary of categories. The report sentiment determination process 156 suitably classifies the outcome of the pathology report 152 as (by way of non-limiting example) non-diagnostic, benign, suspicious or malignant. A concordance or discordance 158 is determined between the one or more first report vocabulary category values and the one or more second report vocabulary category values. A user interface 160 is displayed on a workstation, which presents a comparison of the first medical report 150 and the second medical report 152. In one suitable presentation, the displayed comparison presents the first and second report vocabulary category values and the determined concordance or discordance 158 between the one or more first report vocabulary category values and the one or more second report vocabulary category values.

For example, the illustrative first medical report 150 is a medical imaging examination report, and the one or more first report vocabulary category values may include a BI-RADS score for a Breast Imaging-Reporting (or Radiology) and Data System (BI-RADS) vocabulary category, and a value indicating a tumor biopsy recommendation for a recommendation category. The illustrative second medical report 152 is a pathology report, and the one or more second report vocabulary category values correlating with these vocabulary category values of the imaging report 150 may include a tumor classification value for a tumor classification category. This is merely a non-limiting example and numerous variants are contemplated; for example, in the case of anatomy other than the breast, another Radiology and Data System (RADS) score may be used in place of the BI-RADS score (e.g., a PI-RADS score for the prostate, a LI-RADS score for the lungs, or so forth).

The report tracking task 72 provides a correlation and concordance system between radiology and pathology (or more generally, between reports on two different medical information systems), and leverages both rule-based and statistical machine learning (ML) natural language processing (NLP) components such as the illustrative medical report transform 62 to correlate radiology and pathology reports and evaluate if their sentiment is concordant. The vocabulary of categories 64 enables understanding of important information in the radiology reports, including observations of the physical state of the patient based on radiographic imaging, descriptions of possible diagnosis or interpretations of observations made by the radiologist, and follow-up recommendations for subsequent tests or exams. Properties of these elements are discerned, such as anatomical regions, measurements of observed phenomena, and the vocabulary describing disease or cancer using NLP techniques. The extracted properties correlate to findings, observations, and diagnosis of biopsy procedures described in one or more subsequent and related pathology reports.

Correlation between radiology and pathology reports for a single patient creates a chain of related discourse elements originating in one or more radiology reports and subsequent pathology reports. By correlating the discourse elements in these reports, the ordering physician or institution will be able to measure the quality of the chain of communication between radiologist, pathologist, and ordering physician.

As seen in FIG. 4, the illustrative report tracking implementation includes the data and information extraction module (e.g. the IDRx 62), which extracts clinical data, and syntactic and semantic linguistic information from radiology and pathology information systems (PIS 24 and RIS 26). A module 156 classifies the sentiment of the pathology report outcome, while a module 154 correlates related radiology and pathology reports. A UI 160 is provided to report on the radiology and pathology report concordance (or discordance) 158.

In one suitable embodiment, the data extraction uses an ensemble of rule-based and statistical machine learning (ML) natural language processing (NLP) techniques. Report correlation 154 may rely upon date correlation, by identifying date/times of the radiology and pathology exams for patients. These may be extracted from the contents of the respective reports 150, 152, or may be located independently from a database of the respective pathology and radiology information systems 24, 26. The IDRx 62 suitably extracts elements in a radiology report, e.g. as described with reference to FIG. 2. Example of vocabulary category values that can be grouped per radiologic finding include, by way of non-limiting illustrative example: the anatomical region associated with a radiologic finding; measurement(s) associated with the radiologic finding; a suspicion score associated with the radiologic finding; a biopsy recommendation associated with the radiologic finding; and/or so forth. Examines of vocabulary category values that can be extracted from the pathology report include, by way of non-limiting illustrative example: biopsied anatomy; biopsy procedure; final diagnosis; cancer stage; cancer grade; and/or TNM Classification of Malignant Tumours (TNM) value. The pathology report sentiment analysis 156 implements a method for classifying a pathology report outcome as non-diagnostic, benign, suspicious or malignant. The radiology and pathology concordance (or discordance) is determined by leveraging the vocabulary categories of the respective reports 150, 152 in the common integrated diagnostics representation data and syntactic and semantic linguistic information extracted by the data extraction module. A concordance score between a patient's radiology report 150 and pathology report 152 is computed; where a high concordance score indicates a strong contextual match between the radiology and pathology elements. The UI 160 provides for displaying or visualizing a matched pair of radiology and pathology reports 150, 152 based on concordance scores. The clinician can verify the matched pairs and confirm the result in the dashboard or equivalent visualization interface. The report tracking task 72 thus implements a system for classifying a pathology report as concordant or discordant with the radiology reports.

In one embodiment of the report tracking task 72, this system can compute the concordance scores aggregated for all radiology studies in an institution. The concordance scores can be further subdivided for subsets such as by disease (e.g. lung cancer, breast cancer, prostate cancer, liver cancer etc.), by imaging modality used for radiologic diagnosis (e.g. CT, MR, PET, Ultrasound, X-Ray, etc.), by characteristics of the imaging data (e.g. pixel resolution or slice thickness used for the imaging study, field-strength of the MR scanner, etc.), by subspecialty of the radiologist providing the radiology diagnosis, by interventional devices used for conducting the biopsy.

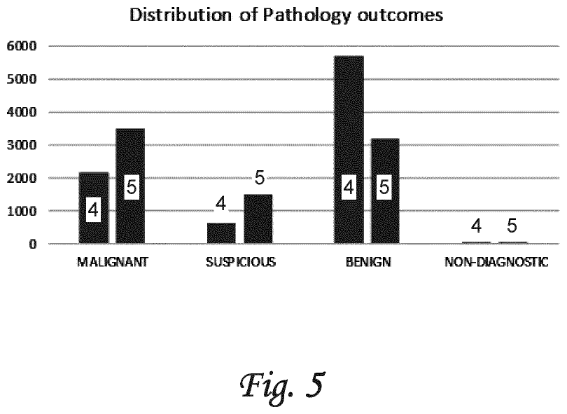

With reference to FIG. 5, as an example a radiology pathology concordance dashboard may display aggregated pathology outcomes for all patients with BI-RADS scores of 4 or 5 (score numbers are labeled on the bars of the plot; BI-RADS score of 4 indicates a suspicious abnormality based on the medical imaging examination; while a BI-RADS score of 5 indicates an abnormality that is highly suspicious of malignancy) a that subsequently underwent a biopsy. The BI-RADS score is an indicator of the risk of malignancy of a breast lesion as interpreted by a radiologist. This distribution of breast pathology outcomes for two BI-RADS risk scores allows an institution to gauge the performance of their breast cancer diagnosis.

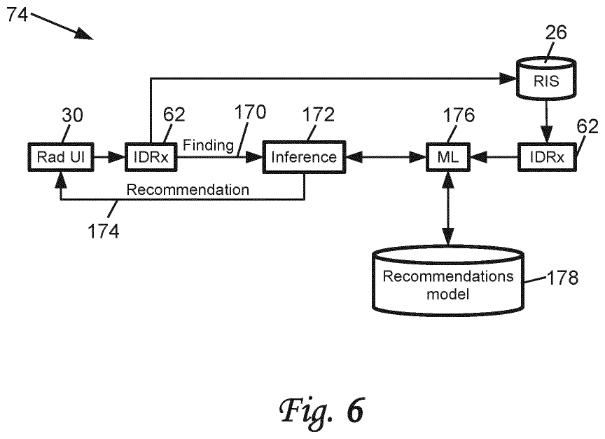

With reference to FIG. 6, the recommendation inference task 74 is defined by instructions stored on the one or more data storage media 14 and executable by the one or more computers 10, 12 to invoke the IDRx 62 to transform text entered into a radiology report via the UI 30 at the radiology workstation 34 connected with the electronic network 16 to the integrated diagnostics representation. A value 170 for a finding vocabulary category is detected in the transformed text. A recommendation inference operation 172 infers a recommendation 174 corresponding to the detected value 170 for the finding vocabulary category using a machine learning (ML) component 176 (as illustrated, or alternatively using a look up table associating recommendations to values for the finding vocabulary category). The inferred recommendation 174 is displayed on the display 40, 42 of the radiology workstation 34. The ML component 176 may, for example, employ a recommendations model 178.

In a typical radiology report format, a (follow-up) recommendation is a statement in the Impressions section of the radiology report in which a radiologist recommends a follow-up exam or test based on analysis of findings in an imaging examination, such as a CT scan, MRI scan, PET scan, or so forth. Typically, findings are explicitly listed in a Findings section that precedes the Impressions section in the radiology report. There is an implicit causal link between a finding and a follow-up recommendation. For example, a finding of a suspicious breast lesion with a BI-RADS score of 4 or 5 typically leads the radiologist to recommend a follow-up biopsy procedure. The language of follow-up recommendations is similar across reports due to the limited vocabulary (captured by the integrated diagnostics representation output by the IDRx 62), and also due to typically terse language used in radiology reports. For example, given a specific type of finding, a range of similar follow-up recommendation statements will exist in a radiology report corpus. The ML component 176 and associated recommendations modell 178 may be implemented as a Natural Language Generation (NLG) component on these relations at scale to automatically generate the follow-up recommendation 174 based on the existence of one or more specific finding(s) 170 in the radiology report under draft. The language of follow-up recommendation statements in impressions sections of radiology reports can vary based on individual radiologists and institutions. The specific language used to make the recommendation and even the decision to make a recommendation at all can be the responsibility of the individual radiologist. An NLG module that suggests or generates an initial framework of responses, ranked by probability, can assist the radiologist in making the decision of making a follow-up recommendation and how best to state that recommendation. The recommendation inference task 74 thus improves the consistency and quality of follow-up recommendations in the clinical workflow. In some embodiments, the ML component 176, 178 is embodied as an NLG module based on Deep Learning (DL) Neural Networks trained on an annotated corpus of past radiology reports with labeled findings and follow-up recommendations that generates candidate follow-up recommendation statements, ranked by probability, to assist a radiology make a decision to include a recommendation statement or not, and guidance on how to state the recommendation. The NLG module based on DL neural network suitably trains on pairs of findings and follow-up recommendations extracted from the past (i.e. historical) radiology reports to create the model 178 of findings and probable recommendation follow-up units of text. In a more specific contemplated implementation, an ensemble of rule-based and statistical ML natural language processing (NLP) components are used to extract relevant units of text.

The recommendation inference task 74 may suitably interact in real time with the report authoring system of the radiology UI 30, by analyzing the text produced by the transcription service as the radiologist is recording their observations (e.g., using the IDRx 62) to detect findings in the radiology report under draft as they are entered in the report. The inference operation 172 then prepares and ranks the most probable follow-up recommendations from the model 178 built by the ML component 176 (e.g. embodied as a DL neural network). The radiology UI 30 may integrate the recommendation inference task 74 as a dedicated window of the UI 30 which lists the recommendation(s) 174 generated by the recommendation inference task 74, and the radiologist may then click on a (follow-up) recommendation displayed in the window (e.g. click using a mouse or other pointing/selection user input device) to have it inserted into the Impressions section of the radiology report under draft. Alternatively, the recommendation 174 may be automatically inserted into the radiology report—however, such an automated approach may not be favored by some radiologists. This may be addressed, for example, by making the optional automatic recommendation insertion operation a user-selectable setting of the UI 30.

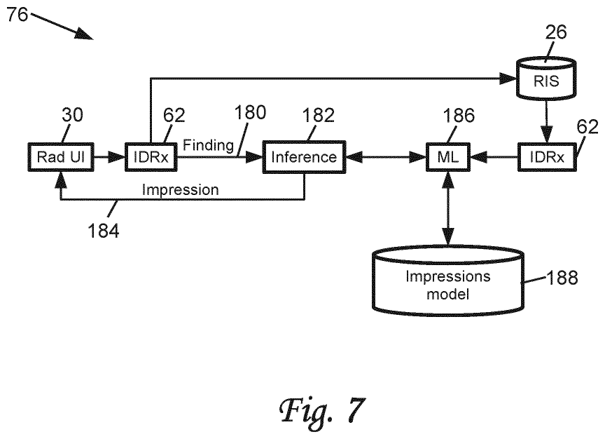

With reference to FIG. 7, the impression inference task 76 is defined by instructions stored on the one or more data storage media 14 and executable by the one or more computers 10, 12 to invoke the IDRx 62 to transform text entered into a radiology report via the UI 30 at the radiology workstation 34 connected with the electronic network 16 to the integrated diagnostics representation. A value 180 for a finding vocabulary category is detected in the transformed text. An impression inference operation 182 infers an impression 184 corresponding to the detected value 180 for the finding vocabulary category using a machine learning (ML) component 186 (as illustrated, or alternatively using a look up table associating impressions to values for the finding vocabulary category). The inferred impression 184 is displayed on the display 40, 42 of the radiology workstation 34. The ML component 186 may, for example, employ an impressions model 188.

Similarly to the ML component 176 and recommendations model 178 of the recommendations inference task 74, the ML component 186 and associated impressions model 188 of the impressions inference task 76 is in some embodiments implemented as a NLP module including rule-based and statistical ML components that extract syntactic and semantic information from the text of the radiology report under draft. Some suitable ML components may include, by way of illustration, a deep learning (DL) neural network module that uses the output of the NLP module (incorporated into the IDRx 62 in the illustrative example) to generate the impressions model 188 that can be used by the ML component 186 to automatically create content for the Impressions section of the radiology report based on transcribed content of the radiology report under draft received from the radiologist. The impressions inference task 76 may suitably interact in real time with the report authoring system of the radiology UI 30, by analyzing the text produced by the transcription service as the radiologist is recording their observations (e.g., using the IDRx 62) to detect findings in the radiology report under draft as they are entered in the report. The inference operation 182 then prepares and ranks the most probable impression(s) from the model 188 built by the ML component 186 (e.g. embodied as a DL neural network). The radiology UI 30 may integrate the impressions inference task 76 as a dedicated window of the UI 30 which lists the impression(s) 184 generated by the impressions inference task 76, and the radiologist may then click on an impression displayed in the window (e.g. click using a mouse or other pointing/selection user input device) to have it inserted into the Impressions section of the radiology report under draft. Alternatively, the impression 184 may be automatically inserted into the radiology report (e.g., if a user-selectable setting of the UI 30 enabling automatic insertion is selected by the radiologist in the UI settings).

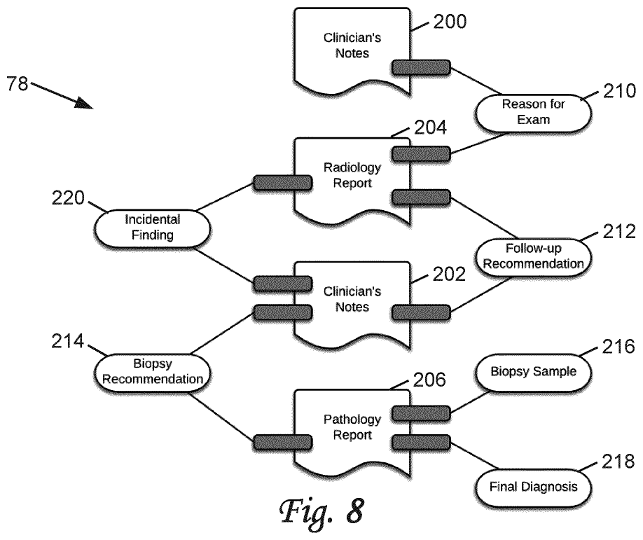

With reference to FIG. 8, an illustrative implementation of the patient timeline task 78 defined by instructions stored on the one or more data storage media 14 and executable by the one or more computers 10, 12 is described. Medical reports on a patient are retrieved from the plurality of medical information systems including, in the illustrative example, a radiology report retrieved from the RIS 26, a pathology report retrieved from the PIS 24, and clinician's notes retrieved from the EHR 22. The IDRx 62 is invoked to transform each retrieved report to the integrated diagnostics representation, as shown in FIG. 8 as: two sets of clinician's notes 200, 202 in the integrated diagnostic report (IDR) format; a radiology report 204 in the IDR format; and a pathology report 206 in the IDR format. Vocabulary category values from different retrieved and transformed medical reports 200, 202, 204, 206 are correlated based on the vocabulary categories of the values and dates of the medical reports. In the illustrative example of FIG. 8, a reason for exam value 210 in the radiology report 204 may be correlated with a value in the clinician's notes 200 describing a clinical observation serving as the basis for the reason for exam 210. The radiology report 204 may further include a follow-up recommendation 212 for a biopsy that correlates with a concurring biopsy recommendation (or biopsy order) 214 in the clinician's notes 202. The biopsy recommendation or order 214 in the clinician's notes 202 may in turn correlate with a biopsy sample 216 and a final diagnosis 218 identified in the pathology report 206. Various other values in the various medical reports 200, 202, 204, 206 may be similarly correlated, such as an incidental finding 220 contained in the radiology report 204 that correlates with content of the clinician's notes 202. The correlation of values in the different medical reports 200, 202, 204, 206 is based on the vocabulary categories of the values (e.g., a value of a recommendation category correlates with a value of a category in a subsequent report describing a test or examination performed in accord with that recommendation) and also based on dates of the medical reports. In illustrative FIG. 8, the reports 200, 204, 202, 206 are dated in that sequence, i.e. the clinician's notes 200 are dated prior to the radiology report 204 which is dated prior to the clinician's notes 202 which are dated prior to the pathology report 206. As an example of using dates for correlation, an examination correlates with a recommendation for that type of examination if the examination occurred after the recommendation (so that the examination is performed in response to the recommendation). A patient timeline for the patient may be displayed, which includes presentation of the correlated vocabulary category values arranged in a temporal sequence in accord with the dates of the medical reports.

To generalize, in some contemplated embodiments, the vocabulary category values from the different retrieved and transformed medical reports 200, 204, 202, 206 are correlated at least in part by correlating a causational vocabulary category value (e.g. a value of a recommendation category) and a responsive vocabulary category value (e.g. a value of a category presenting a medical examination result) based on the combination of (i) the vocabulary category of the responsive vocabulary category value being a response to the vocabulary category of the causational vocabulary category value (here examination result is a response to a recommendation to perform the examination) and (ii) the medical report containing the causational vocabulary category value having an earlier date than the medical report containing the responsive vocabulary category value.

The patient timeline task 78 is based in part on the insight that discourse and pragmatic analysis of clinical text can contribute to a deeper understanding of the intent and goals of the communication of key actors in clinical workflow, such as radiologists, pathologists, and clinicians. Establishing a vocabulary of discourse elements (as per the integrated diagnostics representation, i.e. IDR, output by the illustrative IDRx 62) and how they contribute to the overall discourse structure and pragmatics of clinical reports enables adding an additional layer of semantics to the model of clinical text. The discourse elements in radiology and pathology reports, such as the vocabulary categories of findings, observations, diagnoses, and recommendations, enables identifying, extracting, and presenting an articulated understanding of discourse elements within their clinical report contexts, enabling improved appreciation of their roles and interrelationships across multiple workflow artifacts. Generating the patient timeline entails tracking what diagnostic or therapeutic follow-ups have been recommended for a patient, when and why recommended, and within what time frame. These elements are efficiently captured by the vocabulary category:value elements of the IDR, along with date information contained in the medical reports. These are elements of an evolving treatment plan which can be described by keeping track of the expected consequences of such recommendations and whether and how they have been satisfied or otherwise dispatched in a timely manner. These kinds of inquiries and decisions and requests and responses form an evolving healthcare discourse, and much of the documentation of these behaviors and reasoning is documented within text-based clinical reports. All these discourse elements are extracted by the IDRx 62 and integrated across a chronology of reports to generate a patient timeline for review by medical personnel, and/or for presentation to the patient. The patient timeline task 78 uses as training data an annotated corpora of medical reports labeled with the discourse element vocabulary extracted by the IDRx 62, and ML models are trained on the annotated corpora and used to extract discourse elements from clinical text.

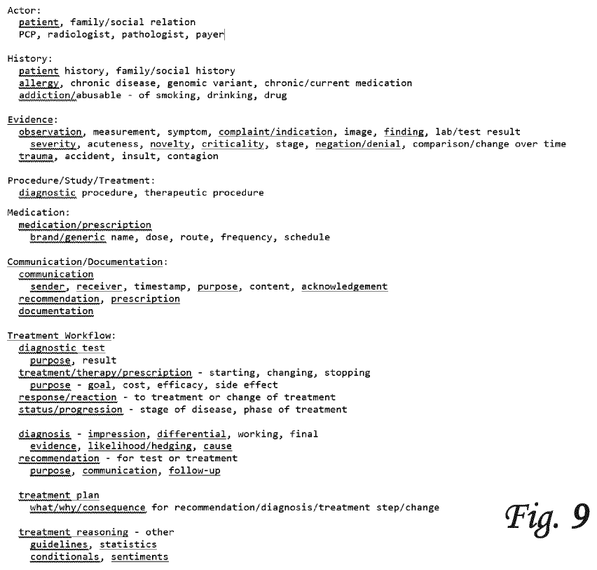

With reference to FIG. 9, a vocabulary outline of discourse elements and pragmatic attributes is shown, which illustrates knowledge structure and relationships among discourse elements and more standard physical entities. In the vocabulary outline of FIG. 9, those elements and attributes that most heavily serve discourse/pragmatic roles are underlined. The full range of medical discourse/pragmatic concepts includes, but is not limited to those underlined, and FIG. 9 is to be understood as a non-limiting illustrative example.

The training of the ML components of the patient timeline task can be performed as follows. A clinical corpora of medical reports is analyzed to create a formal vocabulary of clinical discourse elements and their attributes. The clinical corpora are annotated with formal discourse vocabulary, in collaboration with clinical domain experts, and the ML models are designed and trained. In use, discourse elements are identified and extracted from clinical text (after conversion to IDR format). The discourse element identification and extraction is integrated with Natural Language Understanding (NLU) applications. Discourse elements are linked and connected across reporting artifacts based on a discourse plan or strategy in order to query with natural language a patient's diagnostic record answering questions such as: What was the reason for exam? What treatment did the clinician recommend? Was a biopsy performed? And so forth.

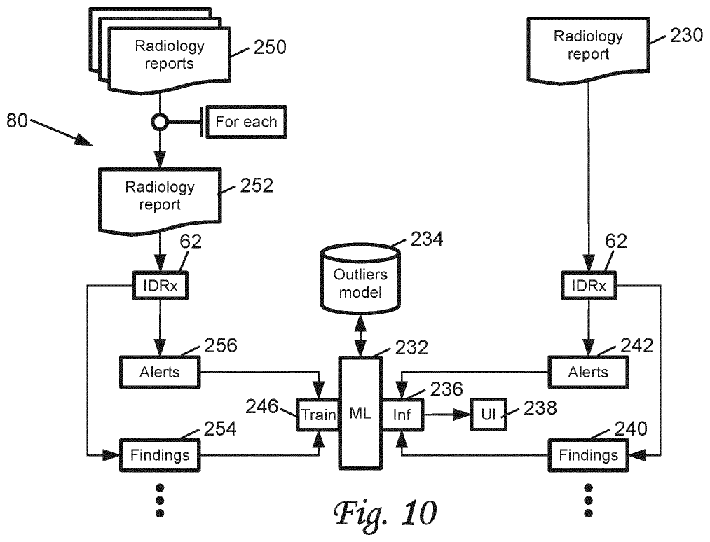

With reference to FIG. 10, an illustrative embodiment of the outliers detection task 80 is described. The outliers detection task 80 is defined by instructions stored on the one or more data storage media 14 and executable by the one or more computers 10, 12 to: invoke the IDRx 62 to transform text entered into a medical report 230 at a workstation connected with the electronic network 16 to the integrated diagnostics representation and detect vocabulary category values in the transformed text (e.g., transform text entered into a radiology report via the UI 30 at the radiology workstation 34 connected with the electronic network 16 to the integrated diagnostics representation); infer a missing or inconsistent vocabulary category value of the medical report by inputting the detected vocabulary category values to an ML component 232, 234 trained to detect missing or inconsistent vocabulary category values in medical reports; and display the inferred missing or inconsistent vocabulary category value on a display of the workstation (e.g. on the display 40, 42 of the radiology workstation 34 in the case of outlier detection in conjunction with a radiology report). More generally, as diagrammatically shown in FIG. 10, an inference operation 236 is performed using the ML component 232, 234 to detect missing or inconsistent vocabulary category values in medical reports, and the content is displayed via a UI 238 as generically shown in FIG. 10 (e.g., the UI 238 may be the radiology UI 30 of FIG. 1 in the case of radiology report processing).

The outliers detection task 80 may be used to detect various types of outliers. In some embodiments, it is used to detect outliers in which a critical finding is not identified in a radiology report as being a critical finding (i.e., its criticality is not properly noted). A critical finding in a radiology image is one that requires a physician's immediate attention. The radiologist who discovers the finding will document it in the findings section of a radiology report and include a critical alert (sometimes referred to by other similar nomenclature such as a critical notification). The alert is manifest in the report by its inclusion in a critical alert section, paragraph, or sentence, with an optional restatement of the critical finding and mandatory documentation of prompt communication with the ordering physician. The section placement, format, and prominence of the alert may or may not be institutionally specified or well-controlled. However, standard radiology practice strictly requires the reporting of an adequate alert of criticalness and documentation of prompt communication, if and only if one or more findings is a critical finding.

A typical institutional radiology reporting protocol may proceed as follows. First, given the purpose of the image assessment, all noteworthy events found in a radiology image are listed and described in the report. Next, given the reported findings, decisions are made as to whether any finding is a critical finding that also warrants a critical alert. This decision is made in accord with medical standards, and particular institutional requirements, and specialized medical expertise. Then, if at least one of the reported findings is critical, the radiology report should document explicitly both the presence of a critical finding and the radiologist's escalated communication with the ordering physician about it. On the other hand, if no reported finding is critical, the report has no such critical alert.

With continuing reference to FIG. 10, which illustrates the outliers detection task 80 operating to detect outliers relating to critical findings, the ML component 232, 234 serves as a critical finding and alert analysis module to assess whether any reported finding 240 is critical, and on that basis whether an alert is called for and present in the radiology report. The ML component 232, 234 identifies and flags outliers, where a report lists a finding 240 the ML component 232, 234 deems critical but contains no alert (or an insufficiently prominent alert), or a radiology report lists no finding the ML component 232, 234 deems critical, but does contain an alert 242. Some types of outliers relating to a critical finding that may be detected by the outliers detection task 80 may include, for example: a radiology report, or an excessive proportion of a set of reports, documents a finding the ML component 232, 234 deems critical along with one of the following: (i) but the report contains no alert/notification; (ii) and the report explicitly documents the presence of a critical finding, but does not document communication with the ordering physician; (iii) and the report documents communication with the ordering physician, but does not explicitly document the presence of a critical finding; and/or (iv) but documentation of either the presence of a critical finding or its communication with the ordering physician is non-standard, not prominent, or otherwise inadequate. For outliers of type (iv), some possible inadequacies include a critical notification that does not contain all of the following key elements—time-frame, person notified, whether within the needed time interval (1 hour, 1 day, etc.), any acknowledgement from receiving entity.

Other types of outliers relating to a critical finding that may be detected by the outliers detection task 80 may include a radiology report, or an excessive proportion of a set of reports, that documents a finding the radiologist deems critical (as indicated by a critical alert contained in the report), but the ML component 232, 234 deems that no finding in the report warrants the included critical alert.

In general, the outliers detection task 80 may be applied in an online mode to assist a radiologist while drafting a radiology report, and/or may be applied in a batch mode to process past radiology reports in order to detect questionable reports and analyze aggregate reporting behavior by radiologist or by institution. While the application to assessing handling of critical findings in radiology reports is described as an illustrative example, the outliers detection task 80 is more generally applicable, e.g. to any text-based medical report in which a (human) clinician assessing medical evidence is required not only to document his/her findings but also to call out, specially communicate, or otherwise escalate attention and response to any critical findings.

With continuing reference to FIG. 10, the ML component 232, 234 is suitably trained, in a training operation 246, on a set (i.e. corpus) of training medical reports 250. In one training approach, for each medical report 252 in the sample corpus 250, the report is processed by the IDRx 62 and findings 254 and critical alerts 256 are extracted. Annotators are applied to demarcate and label each alert region with its kind; each finding with its level of criticalness; and each formal document section as +/−Findings. Boundaries of alert regions are established as follows. For each kind-labeled alert/notification region: the region's report offsets are widened backwards and forwards until they hit the nearest sentence, paragraph, or section boundaries. (configurable for tuning performance); resulting widened spans may overlap with any other report regions; overlaps among alerts/notifications are thereby multiply labeled as to kind. Advantageously, this illustrative approach is a robust, tunable method for titrating the verbiage that is most responsive to, but most ineligible for, predictive modeling from N-gram differentials.

The report 252 is flagged as HasCriticalFinding if and only if at least one of the findings has a “critical” level label. If, across the board, no annotations for findings are provided, this flag is ignored. The report 252 is flagged as HasCriticalAlert if and only if at least one of the alert regions has a “critical” kind label. The report is then trisected into three sets of text regions: all widened alert/notification regions, of all kinds; all +Findings sections, excluding all widened alert/notification regions; and all other sections, excluding all widened alert/notification regions. (This amounts to −Findings plus any remaining report text deemed subject to profiling.) Advantageously, in this illustrative approach the +/−Findings split of sections, and their relative feature weightings, are configurable, thereby enabling more robust model tuning across different report formats. Optionally, the annotated findings may be forcibly included or excluded from each of the three sets of regions. (configurable for regression analyses with/without text of findings themselves). This advantageously assesses and optimizes predictive discrimination even in the absence of annotated findings, which helps mitigate the cost of annotating individual findings, yet boosts discriminative power in their presence. For each set of text regions in the report trisected as just described, the set of widened alert regions is ignored, and only the other two sets of regions are profiled. The regions are profiled by counting the varieties of word N-grams within them, such as all N-grams of width 1, 2, or 3. Each report thus optionally yields a +/−HasCriticalFinding flag, and always yields a +/−HasCriticalAlert flag, and an N-gram-featured profile for each of the +Findings and Other sets of text regions.

The outliers model 234 of the ML component 232, 234 may be constructed as a corpus-populated, N-gram-featured, differential model as follows. The per-report profiles of text regions are aggregated, grouped by +/−HasCriticalFinding (optional), +/−HasCriticalAlert, and +Findings versus other regions. The aggregation is not only of N-gram counts, but also their variance across reports, separately for each N-gram being counted. The +/−HasCriticalAlert discriminative models are derived, optionally 4-way by composing with +/−HasCriticalFinding. Statistical power is optimized by heeding not only differences in N-gram counts, but also differences in count variances and numbers of reports per group. One such model, as an illustrative example, weights signed feature differences with Welch's t-test probabilities. Another such model, as another illustrative example, is one constructed for multinomial logistic regression. Other models, such as soft-margin classifiers, may be constructed from the same data analyses and provide comparable discriminative power. Advantageously, this targeted attention to fine-grained distributional parameters helps mitigate the uneven preponderance of non-critical versus critical reports, and of correct versus faulty reports. As a further advantage, when the uneven distributions of cases are made tractable, the huge preponderance of correct reports becomes highly leverageable. These models effectively leverage a body of radiologists as highly-trained, expert human annotators of the criticalness of findings. Moreover, the impact of the possible presence of a few faulty reports is minimal. The illustrative approach just described helps mitigate the cost of annotating the criticalness of findings, by effectively harvesting past expert annotations from the same institution. The resulting outliers models 234 may be inspected, visually and statistically, for the quality and pertinence of ordered differences in N-gram feature distributions, thus providing developers access to human-interpretable model features. Prominent appearance of model-ineligible alert verbiage may also serve as feedback on possible omission of alert annotators. Advantageously, the systematic use of this feedback from N-grams affords a self-correction mechanism which improves annotator and model quality and robustness. The configurable or otherwise tunable parameters may be tuned to optimize performance on a validation set.

A more specific illustrative embodiment of the inference operation 236 may be performed using the just-described trained outliers model 234 as follows. Given the radiology report 230 to be assessed and classified, it is analyzed by the IDRx 62 and processed analogously to the processing of the training report 252, e.g. using the discriminative model, thus yielding N-gram-featured profiles of report regions of the report 230, a +/−HasCriticalAlert flag, and optionally a +/−HasCriticalFinding flag. Given this report analysis, it is assessed as to: (A) whether this report +/−HasCriticalAlert, and (B) to compare against the corpus-populated differential model 234 to predict whether it should have a critical alert (but does not, and hence is an outlier). The values of this assessment and this prediction serve to classify the report in signal-detection terms, that is, by assigning the report 230 to one of the following classes:
Class True Positive: the report correctly has a critical alert;
Class True Negative: the report correctly omits a critical alert;
Class False Positive: the report incorrectly has a critical alert; or
Class False Negative: the report incorrectly omits a critical alert
   In this classification scheme, a report that is classified with a “False” label (that is, Class False Positive or Class False Negative) is an outlier.

In the above outlier detection approach, estimations of classification goodness of fit are advantageously available. One suitable estimation of classification goodness is the direct N-gram profile similarity metrics, such as cosine similarity. Another suitable estimation of classification goodness is the feature weights from logistic regression prediction. It may be noted that this same embodiment of the inference operation 236 for using the outliers model 234 may also be used for validating and tuning it during construction, to optimize performance against a validation set.

In one application, with the above classifier embedded in a radiology report editor (e.g., by having the radiology UI 30 of FIG. 1 invoke the outliers detection task 80), the classifier suitably assists in drafting the radiology report. While a radiologist is recording findings as part of drafting the report, the classifier can be invoked to recommend appropriate critical alerts in response to entering a finding (as, unless/until the radiologist enters the alert into the report under draft, it is an “outlier”), or highlight any remaining apparent discrepancies between findings and alerts as an automatic check performed on a completed radiology report before it is finalized and stored at the RIS 26. The outliers detection task 80 may also point out the absence of certain key elements in a notification. The intelligence from the classifier trained on the corpus 250 as described above may be used alone or in collaboration with other available expert recommendations or information, such as from RadPeer scoring.

In another (not mutually exclusive) application, with the above classifier may be used in a retrospective batch mode, to identify and flag discrepant reports, and in turn to profile the criticalness thresholds of different institutions, different departments, and different radiologists, and over different time spans. Such thresholds may be compared with institutional guidelines to assess both adherence to guidelines and the adequacy of the guidelines themselves.

In another (not mutually exclusive) application, with the above classifier may be used in a retrospective batch mode to populate groomed lists of different types of discovered findings and their levels of criticalness according to different institutions, departments, or radiologists.

The invention has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A medical information technology (IT) system comprising:
one or more computers; and
one or more data storage media;
wherein the one or more computers and the one or more data storage media are interconnected by an electronic network; and
wherein the one or more data storage media store instructions executable by the one or more computers to define:
a plurality of medical information systems storing medical reports in different respective medical information system-specific medical report formats; and
an integrated diagnostics system including:
a medical report transform operative to transform text of medical reports stored in the different respective system report formats to an integrated diagnostics representation which represents the text of the medical reports as vocabulary category values of a vocabulary of categories; and
a plurality of document processing tasks each operative to invoke the medical report transform to transform one or more medical reports processed by the task to the integrated diagnostics representation and to perform the task on the vocabulary category values of the integrated diagnostics representation of the one or more medical reports processed by the task.

2. The medical IT system of claim 1 wherein the stored instructions are further executable by the one or more computers to define a Picture Archiving and Communication System (PACS) storing medical images, and the plurality of integrated diagnostic tasks include a medical image tagging task defined by instructions stored on the one or more data storage media and executable by the one or more computers to:
identify a medical imaging examination report on a medical imaging examination of a patient;
invoke the medical report transform to transform the identified medical imaging examination report to the integrated diagnostics representation; and
add metadata describing one or more vocabulary category values of the identified and transformed medical imaging examination report to an image of the medical imaging examination of the patient stored on the PACS.

3. The medical IT system of claim 2 wherein the instructions defining the medical image tagging task are further executable by the one or more computers to:
identify a pathology report on the patient;
invoke the medical report transform to transform the identified pathology report to the integrated diagnostics representation;
locate one or more correlated vocabulary category values in the identified and transformed pathology report that correlate with the one or more vocabulary category values described by the metadata added to the image; and
add further metadata to the image describing the one or more correlated vocabulary category values in the identified and transformed pathology report.

4. The medical IT system of claim 2 wherein the added metadata includes at least one hyperlink to the identified report that is transformed to obtain the one or more vocabulary category values described by the metadata.

5. The medical IT system of claim 1 wherein the plurality of integrated diagnostic tasks include a report tracking task defined by instructions stored on the one or more data storage media and executable by the one or more computers to:
identify a first medical report on a patient in a first medical information system which is stored in a first system report format;
invoke the medical report transform to transform the first medical report to the integrated diagnostics representation;
identify one or more first report vocabulary category values in the transformed first medical report;
identify a second medical report on the patient in a second medical information system which is stored in a second system report format that is different from the first system report format;
invoke the medical report transform to transform the second medical report to the integrated diagnostics representation;
identify one or more second vocabulary category values in the transformed second medical report correlating with the first report vocabulary category values;
determine a concordance or discordance between the one or more first report vocabulary category values and the one or more second report vocabulary category values; and
display, on a workstation, a comparison of the first medical report and the second medical report wherein the displayed comparison presents the first and second report vocabulary category values and the determined concordance or discordance between the one or more first report vocabulary category values and the one or more second report vocabulary category values.

6. The medical IT system of claim 5 wherein:
the first medical report is a medical imaging examination report the one or more first report vocabulary category values include a RADS score for a Radiology and Data System (RADS) vocabulary category and a value indicating a tumor biopsy recommendation for a recommendation category;
the second medical report is a pathology report, the one or more second report vocabulary category values include a tumor classification value for a tumor classification category; and
the determined concordance or discordance indicates concordance or discordance between the RADS score and the tumor classification value.

7. The medical IT system of claim 1 wherein the plurality of integrated diagnostic tasks include a recommendation inference task defined by instructions stored on the one or more data storage media and executable by the one or more computers to:
invoke the medical report transform to transform text entered into a radiology report at a radiology workstation connected with the electronic network to the integrated diagnostics representation;
detect a value for a finding vocabulary category in the transformed text;
infer a recommendation corresponding to the detected value for the finding vocabulary category using a machine learning component or look-up table associating recommendations to values for the finding vocabulary category; and
display the inferred recommendation on a display of the radiology workstation.

8. The medical IT system of claim 1 wherein the plurality of integrated diagnostic tasks include an impressions inference task defined by instructions stored on the one or more data storage media and executable by the one or more computers to:
invoke the medical report transform to transform text entered into a radiology report at a radiology workstation connected with the electronic network to the integrated diagnostics representation;
detect a value for a finding vocabulary category in the transformed text;
infer an impression corresponding to the detected value for the finding vocabulary category using a machine learning component or look-up table associating impressions to values for the finding vocabulary category; and
display the inferred impression on a display of the radiology workstation.

9. The medical IT system of claim 1 wherein the plurality of medical information systems includes at least a Pathology Information System (PIS) storing pathology reports in a pathology report format and a Radiology Information System (RIS) storing medical imaging examination reports in a medical imaging examination report format that is different from the pathology report format, and the plurality of integrated diagnostic tasks include a patient timeline task defined by instructions stored on the one or more data storage media and executable by the one or more computers to:
retrieve medical reports on a patient from the plurality of medical information systems including from at least the PIS and the RIS;
invoke the medical report transform to transform each retrieved report to the integrated diagnostics representation;
correlate vocabulary category values from different retrieved and transformed medical reports based on the vocabulary categories of the values and dates of the medical reports; and
display a patient timeline for the patient including presenting the correlated vocabulary category values arranged in a temporal sequence in accord with the dates of the medical reports.

10. The medical IT system of claim 9 wherein the vocabulary category values from the different retrieved and transformed medical reports are correlated at least in part by correlating a causational vocabulary category value and a responsive vocabulary category value based on the combination of (i) the vocabulary category of the responsive vocabulary category value being a response to the vocabulary category of the causational vocabulary category value and (ii) the medical report containing the causational vocabulary category value having an earlier date than the medical report containing the responsive vocabulary category value.

11. The medical IT system of claim 1 wherein the plurality of integrated diagnostic tasks include a outliers detection task defined by instructions stored on the one or more data storage media and executable by the one or more computers to:
invoke the medical report transform to transform text entered into a medical report at a workstation connected with the electronic network to the integrated diagnostics representation and detect vocabulary category values in the transformed text;
infer a missing or inconsistent vocabulary category value of the medical report by inputting the detected vocabulary category values to a machine learning component trained to detect missing or inconsistent vocabulary category values in medical reports; and
display the inferred missing or inconsistent vocabulary category value on a display of the workstation.

12. The medical IT system of claim 11 wherein the medical report comprises a radiology report, the machine learning component includes an outliers model, and the machine learning component is trained to detect:
(i) outliers in which the radiology report includes a finding classified as a critical finding by the outliers model and the radiology report does not include a corresponding critical alert, and
(ii) outliers in which the radiology report includes critical alert corresponding to a finding that is not classified as a critical finding by the outliers model.

13. The medical IT system of claim 1 wherein the integrated diagnostics system transforms a medical report to the integrated diagnostics representation by operations including:
segmenting the medical report into sections;
performing natural language processing to parse text content of each section into tokens; and
matching tokens to vocabulary categories of the vocabulary of categories and deriving vocabulary category value from the tokens and the matched vocabulary categories.

14. The medical IT system of claim 1 wherein the vocabulary of categories include at least “finding”, “critical finding”, “recommendation”, “biopsy sample”, “reason for exam”, “diagnosis”, “impression”, and “observation” vocabulary categories.

15. The medical IT system of claim 1 wherein the stored instructions are further executable by the one or more computers to define a Picture Archiving and Communication System (PACS) storing medical images, and the plurality of medical information systems includes at least a Radiology Information System (RIS) storing medical imaging examination reports in a medical imaging examination report format that is different from the pathology report format, and the medical IT system further comprises:
a plurality of medical imaging devices including at least one magnetic resonance imaging (MRI) scanner, at least one computed tomography (CT) scanner, and at least one positron emission tomography (PET) scanner;
at least one radiology workstation comprising a display and at least one user input device;
the plurality of medical imaging devices connected by the electronic network to transfer medical images acquired by the plurality of medical imaging devices to the PACS; and
the at least one radiology workstation connected by the electronic network to retrieve medical images from the PACS, to display the retrieved medical images on the display of the at least one radiology workstation, to receive an imaging examination report via the at least one user input device of the radiology workstation, and to store the received imaging examination report in the imaging examination report format at the RIS.

16. The medical IT system of claim 1 wherein the plurality of medical information systems includes at least one of a Pathology Information System (PIS) storing pathology reports in a pathology report format and a Radiology Information System (RIS) storing medical imaging examination reports in a medical imaging examination report format that is different from the pathology report format.

17. A non-transitory storage medium storing instructions which are readable and executable by one or more computers to:
extract textual content from a medical imaging examination report on a medical imaging examination of a patient; and
add metadata describing the textual content extracted from the medical imaging examination report to an image of the medical imaging examination of the patient;
wherein the added metadata describing the textual content extracted from the medical imaging examination report includes a hyperlink to the medical imaging examination report;
wherein the instructions are further readable and executable by the one or more computers to:
identify a pathology report on the patient;
extract textual content from the pathology report that correlates with the textual content extracted from the medical imaging examination report; and
add metadata to the image describing the textual content extracted from the pathology report to the image of the medical imaging examination of the patient;
wherein the added metadata describing the textual content extracted from the pathology report includes a hyperlink to the pathology report.

18. (canceled)

19. The non-transitory storage medium of claim 17 wherein:
the textual content extracted from the medical imaging examination report identifies a pathology recommendation contained in the medical imaging examination report, and
the textual content extracted from the pathology report is responsive to the pathology recommendation contained in the medical imaging examination report.

20. The non-transitory storage medium (14) of claim 17 wherein the added metadata are Digital Imaging and Communications in Medicine (DICOM) metadata and the instructions are further readable and executable by the one or more computers to store the image in a Picture Archiving and Communication System (PACS) annotated with the DICOM metadata.

21. A method performed in conjunction with a Pathology Information System (PIS) which stores pathology reports in a pathology report format and a Radiology Information System (RIS) which stores medical imaging examination reports in a medical imaging examination report format that is different from the pathology report format, the method comprising:
using an electronic processor programmed by instructions stored on a non-transitory storage medium;
converting at least one pathology report and at least one medical imaging examination report to an integrated diagnostics representation which represents the text of the converted reports as vocabulary category values of a vocabulary of categories;
temporally ordering the converted reports based on timestamps of the respective reports;
identifying a responsive report and a causational report based on vocabulary category values of the converted responsive report being responsive to vocabulary category values of the converted causational report; and
displaying, on a workstation, a summary of the vocabulary category values used in the identifying.

22. The method of claim 21 wherein the summary includes a timeline representing the causational report and the responsive report wherein each of the causational report and the responsive report is labeled with the vocabulary category values of the respective reports used in the identifying and the timestamps of the respective reports.

23. The method of claim 21 further comprising:
using the electronic processor programmed by instructions stored on the non-transitory storage medium, determining a concordance or discordance between the vocabulary category values of the causational and responsive reports used in the identifying;
wherein the summary includes the determined concordance or discordance.

24. The method of claim 21 further comprising:
adding metadata to an image of a medical imaging examination that is reported upon in the at least one medical imaging examination report, the added metadata describing the vocabulary category values of the causational and responsive reports used in the identifying.

25. The method of claim 21 further comprising:
adding metadata to an image of a medical imaging examination that is reported upon in the at least one medical imaging examination report, the added metadata including hyperlinks to the causational and responsive reports.
